(19) United States
(12) Patent Application Publication
(10) Pub. No.: US 2014/0011711 A1
(43) Pub. Date: Jan. 9, 2014

(54) MICROARRAY CELL CHIP

(71) Applicant: Solidus Biosciences, Inc., (US)

(72) Inventors: Moo Yeal Lee, Burlingame, CA (US); Dong Woo Lee, Suwon-Si (KR); Se Hoon Jeong, Seongnam (KR); Jeong Suong Yang, Yong-in Gu (KR); Bo Sung Ku, Suwon-Si (KR)

(73) Assignee: Solidus Biosciences, Inc., San Francisco, CA (US)

(21) Appl. No.: 13/870,443

(22) Filed: Apr. 25, 2013

Related U.S. Application Data
(63) Continuation of application No. 13/228,668, filed on Sep. 9, 2011, now abandoned.
(60) Provisional application No. 61/381,812, filed on Sep. 10, 2010.

(51) Int. Cl. C12M 1/32

(52) U.S. Cl. CPC C12M 23/12
USPC 506/40

(57) ABSTRACT

Disclosed herein is a microarray cell chip. The microarray cell chip includes an upper substrate that has biomatrices encapsulating biomaterials formed on one surface thereof and through holes penetrating from one surface to the other surface thereof and a lower substrate that is coupled with the upper substrate and is provided with wells storing reagents supplied to the biomatrices. The microarray cell chip according to the present invention can smoothly transfer the culture media and the reagents to the biomaterials embedded in the biomatrices through the diffusion and simply separate the upper substrate from the lower substrate, thereby improving the easiness of washing. Therefore, the present invention provides an environment similar to the bio environment, thereby making it possible to increase accuracy of an experiment.

### RELATED APPLICATIONS

This application is a continuation of U.S. application Ser. No. 13/228,668, filed Sep. 9, 2011 which claims the benefit of U.S. Provisional Application No. 61/381,812, filed on Sep. 10, 2010. The entire teachings of the above application are incorporated herein by reference.

### BACKGROUND OF THE INVENTION

1. Technical Field

The present invention relates to a microarray cell chip.

2. Description of the Related Art

A process of developing a new drug is complicated. Further, a cell culture should be performed in order to test efficacy and toxicity of new drug candidates. Generally, a cell culture method may be largely classified into a 2D cell monolayer culture that cultures cells by attaching them to a 2D surface and a 3D cell culture that cultures cells by encapsulating them into a 3D biomatrix. 3D cell culture has been generally known as having a more suitable biomatrix environment than that of a 2D cell culture.

2D cultures generally use a microtiter plate (for example, 6-well, 12-well, 24-well, 96-well, 384-well, 1536-well microtiter plate, or the like) in which a plurality of wells are arranged on a plastic plate. The in vitro microtiter plate approach can be used to rapidly perform several multiple parallel experiments at low cost, particularly when compared to animal studies and eventually human clinical trials. However, since the cells are fixed into the wells, it is difficult to wash the wells after contacting with a compound to be screened, such as a drug or drug candidate. In particular, this problem is more serious when the size of the well is reduced and the number of wells is increased in order to perform more than one experiment using one plate such as a 384-well or 1536-well microtiter plate.

In order to perform experiments in a smaller volume and to overcome this problem, an array based chip can be used, in which cells are encapsulated on a surface-treated glass substrate. The encapsulated cells are in a 3D environment. This approach has been developed by Solidus Biosciences, Inc. See U.S. Ser. No. 12/091,990, which is incorporated herein by reference. The experiments were performed by forming collagen or alginate microdroplets encapsulating cells on the glass substrate in an array format without the presence of specific wells. Preferred chips place biomatrices having cells therein on a flat substrate, thereby making it possible to rapidly perform the toxicity test of a drug while more easily performing washing and reducing the volume, as compared to the existing microtiter plate. However, since the array based cell chip is implemented on the plate, it is more likely to lead to experimental errors due to the very small amount of liquid (containing the test compound) added evaporates by being exposed to air. As a result, it is not suitable to observe cells for a long period of time (for example, greater than one day) even where high humidity is maintained.

### SUMMARY OF THE INVENTION

The present invention has been made in an effort to provide a microarray cell chip, which includes a lower substrate formed with wells in which culture media or reagents (further including drugs and drug candidates and other test compounds as well as enzymes, DNA, RNA, antibodies, viruses, or the like) supplied to cells are stored and an upper substrate formed with biomatrices that encapsulate and culture cells and directly transferring the culture media or the reagents in the wells of the lower substrate to the cells by combining the upper substrate with the lower substrate.

Further, the present invention has been made in an effort to basically prevent cross contamination due to a mixing of culture media or reagents since the culture media or the reagents are included in wells of a lower substrate and can easily perform washing of the cells since only an upper substrate is separated when it is necessary to wash cells. Further, the present invention has been made in an effort to test the influence of reagents while culturing cells for a long period of time since the influence of the evaporation is minimized due to a sufficient amount of culture media included in wells of a lower substrate.

In addition, the present invention has been made in an effort to form through holes on an upper substrate in order to solve a bubble problem that may be caused in wells of a lower substrate when cells are cultured for a long period of time by combining an upper substrate with a lower substrate.

In addition, the present invention has been made in an effort to provide an environment similar to a human or an animal by encapsulating cells having a micro volume to rapidly test a mechanism of efficacy and toxicity of drugs and properly modify several reagents (for example, performing experiments by modifying cells to meet a specific purpose through gene transfection or RNA interface), thereby making it possible to accurately secure predictable toxicity data and replace complex and expensive human/animal clinical trials.

In one embodiment, a microarray cell chip comprising an upper substrate that has biomatrices encapsulating biomaterials formed on one surface thereof and one or more through-holes penetrating from one surface to the other surface thereof. In the upper substrate microarray, at least some through-holes are positioned to engage a corresponding well of a lower substrate. At least some of the through-holes are in near proximity to at least one deposit of biomatrix encapsulating biomaterials. The deposit and the through-holes can be engaging one microwell from a corresponding lower substrate.

A microarray cell chip according to a preferred embodiment of the present invention includes: an upper substrate that has biomatrices encapsulating biomaterials formed on one surface thereof and through-holes penetrating from one surface to the other surface thereof The upper substrate may be engaged with a lower substrate that has wells capable of holding fluids which can be brought into contact with biomatrices.

The biomatrices can be prepared from biopolymers, extracellular material or a hydrogel. Preferably, the biomatrice can be a hydrogel matrix, such as collagen or alginate, which supports cell growth at the microscale.

The microarray cell chip further includes an adhesive layer between contacting surfaces of the biomatrices and the upper substrate. In one embodiment, the adhesive substance covalently attaches the biomatrice material to the substrate surface.

The sectional surface of the through-hole can be a polygon, circle or other shape.

The through-hole is adjacently formed at an outer side of the contacting surface of the biomatrices and the upper substrate.

An inlet area of the through-hole formed on one surface of the upper substrate is optionally larger than an inlet area thereof formed on the other surface thereof.

The inlet of the through hole formed on one surface of the upper substrate is positioned on a vertical surface of the well.

In a preferred embodiment, the upper substrate is couple to a lower substrate such that the biomatrices are inserted into a corresponding well in the lower substrate.

The upper substrate and the lower substrate are provided with a protruding portion or lip along one or more edges and a coupling portion which permits coupling or engaging them to each other.

The microarray cell chip further includes a spacer (or pillar or micro-column) formed on the contacting surface of the biomatrices and the upper substrate. The upper surface of the spacer preferably provides an adhesive layer encapsulating the biomatrices.

The biomatrices preferably include an amine group.

The spacer is preferably formed of a PSMA.

The through hole is preferably adjacently formed or located at the outer side of the spacer.

The biomatrices and the spacer are preferably configured to be inserted into the well upon engaging the lower and upper substrates.

The upper surface of the spacer can be concavely formed from the outer side portion to the central portion and the biomaterial can be collected at the central portion.

The upper surface of the spacer can be formed with a plurality of concave portions and the biomaterials can be collected at the concave portions.

Alternatively, the spacer surfaces can be planar.

The upper substrate and the lower substrate can be provided with a protruding portion and a coupling portion coupling and encapsulating them to each other.

The biomatrices can be formed in plural, having an array form and the wells have the same array form as the biomatrices.

### BRIEF DESCRIPTION OF THE DRAWINGS

FIG. 1 is a cross-sectional view schematically showing a cell chip according to a preferred embodiment of the present invention;

FIGS. 2 to 5 are cross-sectional views showing modified examples of the cell chip shown FIG. 1;

FIG. 6 is a perspective view of the cell chip in which biomatrices and wells are arranged in an array form;

FIGS. 7A and 7B are top images and side images showing a part of a microarray cell chip according to the present invention;

FIGS. 8A and 8B are images generated by being subjected to a toxicity test of reagents using the microarray cell chip according to the present invention; and

FIG. 9 is a scan image generated by being subjected to the toxicity test of reagents by using the microarray cell chip according to the present invention.

### DESCRIPTION OF THE PREFERRED EMBODIMENTS

Various features and advantages of the present invention will be more obvious from the following description with reference to the accompanying drawings.

The terms and words used in the present specification and claims should not be interpreted as being limited to typical meanings or dictionary definitions, but should be interpreted as having meanings and concepts relevant to the technical scope of the present invention based on the rule according to which an inventor can appropriately define the concept of the term to describe most appropriately the best method he or she knows for carrying out the invention.

The above and other objects, features and advantages of the present invention will be more clearly understood from the following detailed description taken in conjunction with the accompanying drawings. In the specification, in adding reference numerals to components throughout the drawings, it is to be noted that like reference numerals designate like components even though components are shown in different drawings. Further, when it is determined that the detailed description of the known art related to the present invention may obscure the gist of the present invention, the detailed description thereof will be omitted.

In one embodiment, a microarray cell chip comprising an upper substrate that has biomatrices encapsulating biomaterials formed on one surface thereof and one or more through-holes penetrating from one surface to the other surface thereof. In the upper substrate microarray, at least some through-holes are positioned to engage a corresponding well of a lower substrate. At least some of the through-holes are in near proximity to at least one deposit of biomatrice encapsulating biomaterials. The deposit and the through-holes can be engaging one microwell from a corresponding lower substrate. In one embodiment, the substrate is glass. The glass substrate can be further functionalized by treatment with 3-(aminopropyl)trimethoxysilane (APTMS) followed by treatment with poly(styrene-co-maleic anhydride) (PSMA). The functionalization can be made on individual spots on the substrate surface or they can be made on the entire/partial surface of the substrate. In one embodiment, the upper substrate is a pillar array. The pillar array can be made of a plurality of microcolumns projecting away from the support structure. The microcolumns or pillars can be spacers. The microcolumns may have a distal end surface that can be used to spot the biomatrice encapusulating biomaterials. The distal surface of the microcolumns can be functionalized by treatment with APTMS and PSMA. In a further embodiment the glass slide may be functionalized with methyltrimethoxysilane (MTMOS). In a further embodiment, APTES (aminopropyltriethoxysilane) may be used in place of APTMS. In a further embodiment, PTMOS (propyltrimethoxysilane) and OTMOS (octyltrimethoxysilane) may be used in place of MTMOS.

The materials and methods (including without limitation, the biomatrices, biomaterials, supports, functionalization, etc.) used in U.S. Ser. No. 12/091,990 to make the chips described therein, which is incorporated herein by reference, can be used to create chips of the present invention.

A microarray cell chip according to a preferred embodiment of the present invention includes: an upper substrate that has biomatrices encapsulating biomaterials formed on one surface thereof and through-holes penetrating from one surface to the other surface thereof The upper substrate may be engaged with a lower substrate that has wells capable of holding fluids which can be brought into contact with biomatrices. In one embodiment, the through-hole is adjacently formed at an outer side of the contacting surface of the biomatrices and the upper substrate.

The lower substrate can be sized to fit the at least one biomatrice encapsulating biomaterials formed on the upper substrate and at least one or more through-holes in close proximity The wells of the lower substrate can optionally contain biomatrices containing Cytochrome P450 (individual isoform or mixture of isoforms) or a small molecule drug, a biopolymer or a combination thereof In one embodiment, a biomatrix containing Cytochrome P450 is deposited within the well surface of the lower substrate. Common P450 isoforms that are applicable are 1A1, 1A2, 1B1, 2A6, 2B6, 2C8, 2C9, 2C18, 2C19, 2D6, 2E1, 2J2, 3A4, 3A5, 3A7, 4B1, 4F8, 4F12, 7B1, 26B1, 27A1, and 39A1. In addition to P450s other Phase I metabolism-based enzymes can be used, including flavin monooxygenases, monoamine oxidases, various esterases, quinone reductases, peroxidases, and alcohol dehydrogenases. In addition to Phase I enzymes, Phase II metabolism-based enzymes can be used, including uridinyl glucuronosyl transferases (particularly isoforms 1A1, 1A3, 1A4, 1A5, 1A6, 1A7, 1A8, 1A9, 1A10, 2B4, 2B7, 2B10, 2B11, 2B15, ad 2B17), epoxide hydrolases, N-acetyl transferases, glutathione S-transferases, sulfotransferases (particularly isoforms 1A1, 2B1a, 2B1b, and 1E1), and catechol O-methyltransferases. In addition to the aforementioned enzymes and their isoforms, a wide range of synthetically relevant enzymes from human and non-human sources can be used, including those contained within Enzyme Commission (EC) Classes 1-6, e.g., Class 1 (oxidoreductases), Class 2 (transferases), Class 3 (hydrolases), Class 4 (lyases), Class 5 (isomerases), and Class 6 (ligases).

In another embodiment, a test compound encapsulated in a biomatrice can be deposited within the well surface of the lower substrate.

The biomatrix can be prepared from biopolymers, extracellular material or a hydrogel. Preferably, the biomatrices can be a hydrogel matrix, such as collagen or alginate, which supports cell growth at the microscale.

The microarray cell chip further includes an adhesive layer between contacting surfaces of the biomatrices and the upper substrate. In one embodiment, the adhesive substance covalently attaches the biomatrix material to the substrate surface.

The sectional surface of the through-hole can be a polygon, circle, or other shape. The through-hole can be any shape or size that allows gases to escape. In one embodiment, the inlet area of the through-hole formed on one surface of the upper substrate is larger than an inlet area thereof formed on the other surface thereof.

In one embodiment, the inlet of the through hole formed on one surface of the upper substrate is positioned on a vertical surface of the well.

In one embodiment, the upper substrate is comprises a protruding portion and the lower substrate comprises a well sized to fit the protruding portion along with at least one through-hole adjacent to the protrusion on the upper substrate. The protrusion of the upper substrate can be part of a pillar array wherein at least some of the pillar surfaces have biomatrix encapsulating biomaterials deposited thereon.

In one embodiment, the microarray cell chip further includes a spacer formed on the contacting surface of the biomatrices and the upper substrate.

In one embodiment, the upper surface of the spacer is further provided with an adhesive layer encapsulating the biomatrices. A preferred spacer is PSMA.

In one embodiment, the biomatrices and the spacer are inserted into the well.

In one embodiment, the upper surface of the spacer is concavely formed from the outer side portion to the central portion and the biomaterial is collected at the central portion. In one embodiment, the upper surface of the spacer is formed with a plurality of concave portions and the biomaterials are collected at the concave portions.

In one embodiment, the upper substrate and the lower substrate are provided with a protruding portion and a coupling portion coupling and encapsulating them to each other.

The biomatrices is formed in plural, having an array form and the wells have the same array form as the biomatrices.

A process for manufacturing a microarray is also described wherein a collagen (or other biomatrix) solution containing mammalian cells is printed atop an island of bottom collagen optionally having a hyaluronan layer deposited thereon.

The volume of the biomatrix containing biomaterial spots may range from about 10-100 nL, from about 20-80 nL or from about 30-60 nL. These samples may be arrayed depending on the size of the substrate and may be in a regular or predetermined pattern. The pattern selected need not be a regular pattern or evenly arrayed. Regular arrays may include 14×40, 20×54, or larger arrayed patterns.

For example, in the case of a 20×54 pattern, 45 regions (5×9) are produced each with a 4×6 array. Larger numbers of spots would necessitate a larger array and creating such larger arrays are contemplated within the invention.

In one embodiment, cell-containing samples with a volume of 30 nL each on a 14×40 spot array deposited on a 25×75 mm² glass slide, the spot diameter is about 0.6 mm (close to the expected size for hemispherical spots), the thickness of approximately 50 μm, and the center-to-center distance of about 1.2 mm is disclosed.

Cells that can be used, or the tissues/organs they can be derived from, include, but are not limited to bone marrow, skin, cartilage, tendon, bone, muscle (including cardiac muscle), blood vessels, corneal, neural, brain, gastrointestinal, renal, liver, pancreatic (including islet cells), lung, pituitary, thyroid, adrenal, lymphatic, salivary, ovarian, testicular, cervical, bladder, endometrial, prostate, vulval, esophageal, etc.

Also included are the various cells of the immune system, such as T lymphocytes, B lymphocytes, polymorphonuclear leukocytes, macrophages, and dendritic cells.

In addition to human cells, or other mammalian cells, other organisms can be used. For example, in testing for environmental effects of an industrial chemical, aquatic microorganisms that could be exposed to the chemical can be used. In still another example, organisms such as bacteria that are genetically engineered to possess or lack a certain trait could be used. For example, in the optimization of an antibacterial lead compound for combating antibiotic resistant organisms, the cell assay could include cells that have been engineered to express one or more genes for antibacterial resistance.

Hereinafter, preferred embodiments of the present invention will be described in detail with reference to the accompanying drawings.

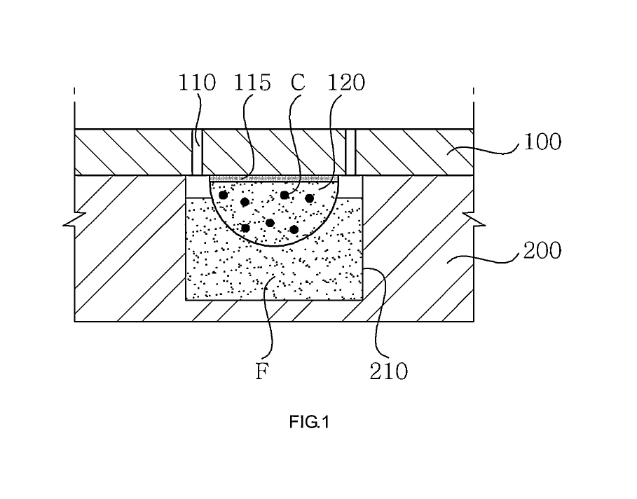

FIGS. 1 to 6 are cross-sectional views and perspective views schematically showing a microarray cell chip according to preferred embodiments of the present invention. A microarray cell chip according to the present invention will be described with reference to the Figures.

As shown in FIG. 1, the microarray cell chip (hereinafter, referred to as a cell chip) includes an upper substrate 100 formed with biomatrices 120 encapsulating a biomaterial C and a lower substrate 200 supplying culture media or reagents (culture media or reagents may be simultaneously supplied, which are referred to as a fluid F) to the biomatrices.

In this configuration, the reagent supplied to wells 210 of the lower substrate 200 may include drugs necessary for specific experiments in order to provide an environment more similar to the bio environment for the biomaterial C as well as a dyed material (for example, fluorescent material and luminescent material), protein, plasmid, DNA, interference RNA, antigen/antibody, virus, or the like.

The biomatrices 120 embeds the biomaterial C. The term “biomaterial” is referred to as various types of biomolecules or biomaterials. An example of the biomolecule may include a nucleic acid arrangement (for example, DNA, RNA, oligo nucleotide, cDNA, extranuclear plasmid, or the like), peptide, protein, fatty, protein or lipid film, organic or inorganic chemical molecule (for example, compound of pharmaceuticals or another fields), virus particles or eukaryotic cell, prokaryotic cell, blast cell component or organelle, or the like.

The biomatrices 120 may be configured to include a sol-gel capable of encapsulating a biomaterial, an inorganic material, an organic polymer, or an organic-inorganic complex material. In particular, it is preferable that the biomatrices 120 may use an extracellular matrix, such as collagen having a porous structure and moving through diffusion of a fluid, or hydrogel without toxicity in a biomaterial such as alginate.

The biomatrices 120 provides environment similar to bio environment to the biomaterial C or provides environment suitable for specific experiments by supplying a fluid to the biomaterial C through diffusion.

The biomatrices 120 having the biomaterial C therein are formed by being spotted to the upper substrate 100 in the state where the biomaterial C and the biomatrices 120 are mixed or may be formed by first spotting the biomatrices 120 and then the biomaterial C thereon. In particular, when the biomaterial C and the biomatrices 120 are formed by being spotted to the upper substrate 100 in the state where they are mixed, the biomaterial C is encapsulated to the biomatrices 120 while being embedded therein.

The upper substrate 100 and the lower substrate 200 are formed of a glass substrate, a plastic substrate, a ceramic substrate, or the like. The shape of the upper substrate 100 and the lower substrate 200 is not limited but the thickness thereof can be optionally controlled.

In addition, an adhesive layer 115 may be further formed between a contact surface in order to increase adhesion between the upper substrate 100 and the biomatrices 120. When the alginate is applied to the biomatrices 120, it is preferable that the adhesive layer 115 is made of a mixture of poly-L-lysine (PLL)-barium chloride. Further, it is preferable to use collagen with good affinity in order to bond the biomatrices 120 made of collagen to the upper substrate 100.

A through hole 110 is formed on the upper substrate 100. The through hole 110 serves as a moving path for bubbles and external air generated by culturing the biomaterial C and contacting and reacting reagents. In addition, when the upper substrate 100 combines with the lower substrate 200 in the state where excessive fluid F is supplied to the well 210 formed on the lower substrate 200, the through hole 110 serves as a path capable of spotting extra fluid F to the outside of the cell chip. The through hole 110 serves to reduce the warpage of the upper substrate 100. In particular, when the large-area upper substrate 100 is used, the upper substrate 100 is consecutively warped from one side to the other side. In this case, the through hole 110 stops the consecutive warpage so as to reduce the warpage of the upper substrate 100.

It is preferable that the through hole 110 is adjacently formed to the outside of the contact surface between the biomatrices 120 and the upper substrate 100. The bubbles generated at the time of the contact and reaction between the biomaterial C and the fluid F are generated around the biomatrices 120. It is preferable that this structure rapidly removes the bubbles.

For the same reason, it is preferable that the through hole 110 of which the inlet is formed with the biomatrices is disposed on a vertical surface of the well 210 formed on the lower substrate 200.

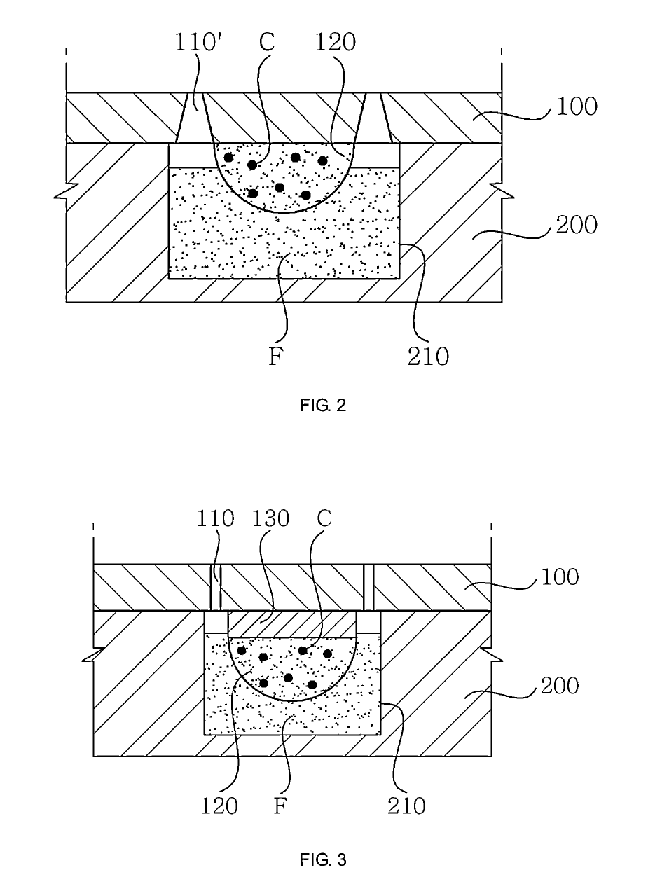

Further, it is preferable that a through hole 110′ has the inlet area formed on one surface of the upper substrate 100 to be larger than the inlet area formed on the other surface thereof; as shown in FIG. 2. To this end, the through hole 110 may be formed to have a truncated shape. When the inlet of one surface formed with the biomatrices 120 is large, it is easy to spot the bubbles and the extra fluid and when the inlet of the other surface is small, it can prevent foreign materials from being introduced from the outside.

The section of the through holes 110 and 110′ may be modified in a polygonal shape.

The lower substrate 200 combined with the upper substrate 100 on which the biomatrices 120 are formed are provided with the well 210 storing the fluid F. The shape of the well 210 is not limited, but it is preferable that the area of the well 210 is larger than the biomatrices 120 so that the biomatrices 120 may be inserted thereinto and the depth thereof is also larger than the height of the biomatrices 120.

The fluid F stored in the well 210 is supplied to the biomatrices 120 inserted into the well 210 and moves to the biomaterial C by the diffusion.

The washing problem having the microtiter plate according to the prior art can be solved by separating the functions of the upper substrate 100 and the lower substrate 200 and the problem of the cross contamination or the drying accompanied by the array based cell chip formed on the existing plate can also be solved. That is, the cell chip according to the present invention can perform the washing in a simple manner that combines the upper substrate 100 with another lower substrate 200 by separating the upper substrate 100 since the biomaterial C is not directly disposed in the well 210 storing the fluid, such that residual materials does not remain in the well 210 and the lower substrate 200 may be reused after the washing, unlike the existing microtiter plate.

The cell chip according to another embodiment of the present invention further includes a spacer 130 formed on the contact surface between the biomatrices 210 and the upper substrate 100, as shown in FIG. 3. The spacer 130 separates the biomatrices 210 from the surface of the upper substrate 100 in order to enable the biomatrices 210 to be completely dipped in the fluid F when the upper substrate 100 is combined with the lower substrate 200. Therefore, the fluid F is supplied to the biomaterial C embedded in the biomatrices 210 under the same conditions.

The configuration of the cell chip according to the preferred embodiment described with reference to FIGS. 1 and 2 may be applied, with being partially modified.

For example, it is preferable that the through hole 110 formed on the upper substrate 100 and serving as the moving path of the bubbles to the outside air to spot the extra fluid F to the outside of the cell chip is adjacently formed to the outside of the spacer 130. It is preferable that the inlet of the through hole 110 is disposed on the vertical surface of the well 210 and the shape of the through hole 110 may be modified as shown in FIG. 2.

In addition, the adhesive layer for encapsulating the biomatrices 210 may be further provided on the upper surface of the spacer 130. When the alginate is applied to the biomatrices 120, it is preferable that the adhesive layer 115 is made of a mixture of poly-L-lysine (PLL)-barium chloride.

In this case, when the spacer is made of polystyrene-co-maleic anhydride (PSMA) and the biomatrices 210 having an amine group are used, the adhesion between the spacer 130 and the biomatrices 210 can be improved even though the separate adhesive layer is not formed on the upper surface of the spacer 130.

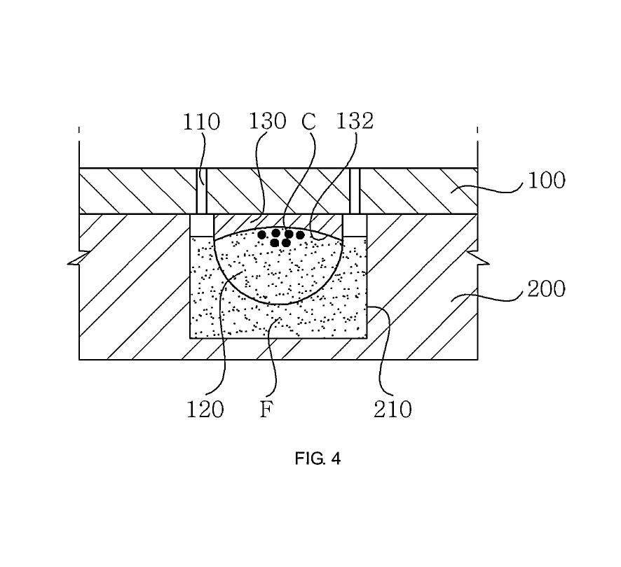

In addition, in the cell chip according to another prepared embodiment of the present invention, the upper surface 132 of the spacer 130 is concavely formed from the outside portion to the central portion and the biomaterials C are collected at the central portion, as shown in FIG. 4.

Since the biomaterials C are not dispersed in the biomatrices 210 but collected in the central portion, the cell chip may measure the influence of the adjacent biomaterials at the time of the contact and reaction of the biomaterial and the fluid (for example, cell-cell interactions). Various bio environments may be reflected to the cell chip by providing the reacting conditions.

The cell chip shown in FIG. 4 may be formed by first spotting the biomaterials C to the upper substrate 100 and then, spotting the biomatrices 210 thereon or may be completed by spotting the biomatrices 210 including the biomaterial C and then, collecting the biomaterials C to the central portion of the upper surface 132 of the spacer 130 by using a centrifugal separator, and combining the upper substrate 100 with the lower substrate 200.

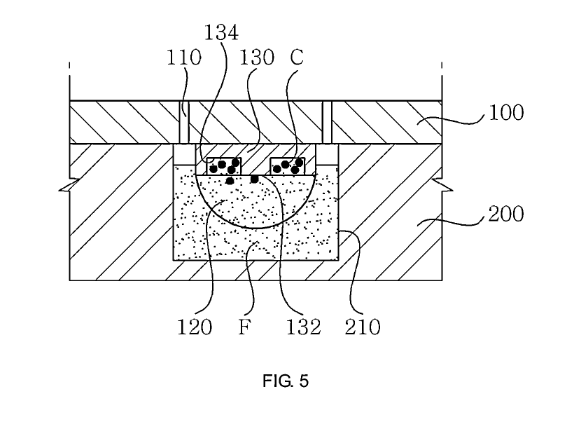

In addition, as shown in FIG. 5, the cell chip may be modified so that the plurality of concave portions 134 are formed on the upper surface 132 of the spacer 130 and the biomaterials C are collected in the concave portion 134.

It is preferable that the cell chip shown in FIG. 5 has the same effect as the cell chip shown in FIG. 4 by collecting the biomaterials C in the concave portion 134 and comparing the reactivity between the biomaterials C collected in the concave portion 134 and the other biomaterials C.

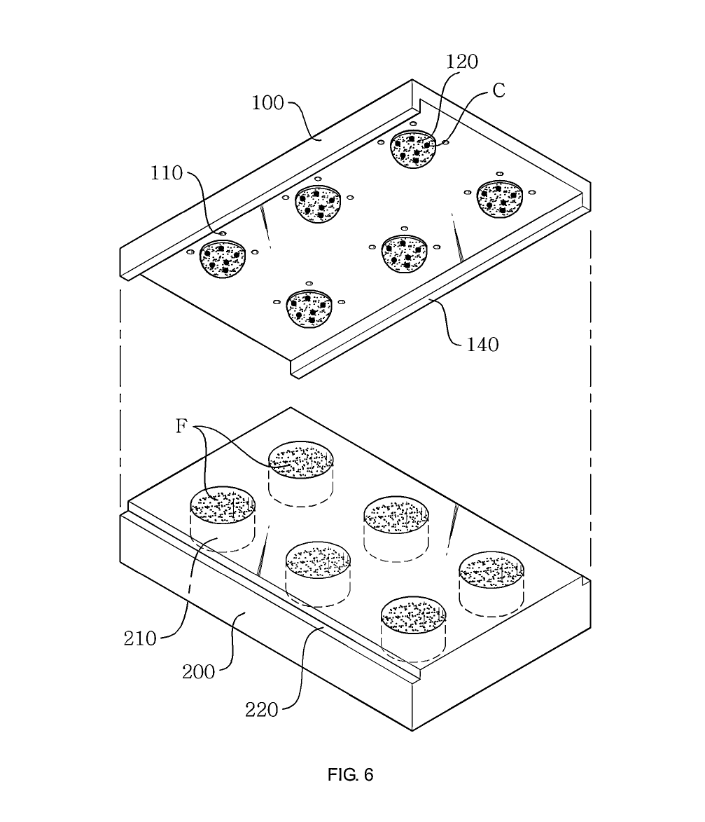

As shown in FIG. 6, in the cell chip according to another preferred embodiment of the present invention, the biomatrices 210 are arranged in an array form and formed in plural and the well 210 has the same arrangement as the biomatrices 210 so that it may be inserted with the biomatrices 120.

The change in the biomaterials C depending on various environments due to the change in the fluid F (in particular, changing reagent) may be observed even though the plurality of biomatrices 210 embedding the same biomaterial C are formed on the upper substrate 100 and the change in the biomaterials C depending on the same environment due to the supply of the same fluid F may be observed even though the biomatrices 210 embedding various biomaterials C are formed on the upper substrate 100.

It is preferable that the cell chip includes protruding portions 140 and combining portions 220 combining and encapsulating the upper substrate 100 and the lower substrate 200 with each other.

FIG. 6 shows a case where a pair of protruding portions 140 is formed on the upper substrate 100 and a pair of combining portions 220 are formed on the lower substrate 200. When the upper substrate 100 is combined with the lower substrate 200, the upper substrate 100 is completely encapsulated to the lower substrate 200 by combining the protruding portion 140 with the combining portion 220 while the biomatrices 210 and the wells 210 are aligned in the array form.

The protruding portion 140 and the combining portion 220 have a configuration to encapsulate the upper substrate 100 and the lower substrate 200 but may be formed in an opposite shape to one shown in FIG. 6 (that is, the protruding portion is formed on the lower substrate and the combining portion is formed on the upper substrate). Therefore, the shape of the protruding portion 140 and the combining portion 220 may be modified. However, it is preferable that the upper substrate and the lower substrate are formed at the edge region so as not to hinder the arrangement of the bio matrices and the wells formed on the upper substrate and the lower substrate.

It is apparent to those skilled in the art that FIG. 6 shows a cell chip based on the cell chip shown in FIG. 1, but the modified shaped shown in FIGS. 2 to 5 may be applied.

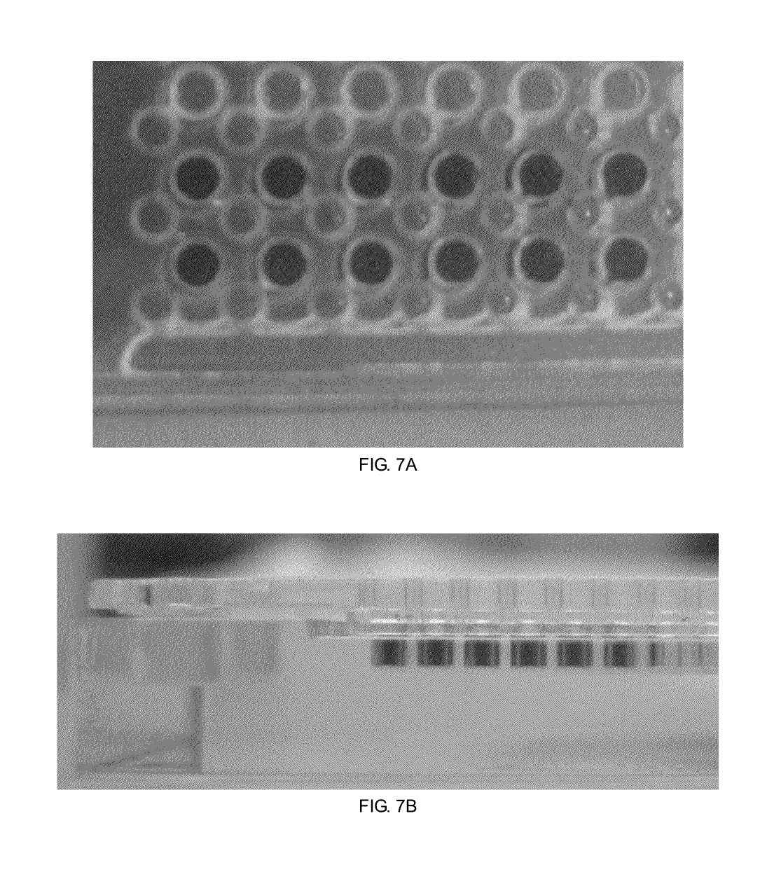

FIGS. 7 to 9 show experiment examples using a microarray cell chip according to the present invention.

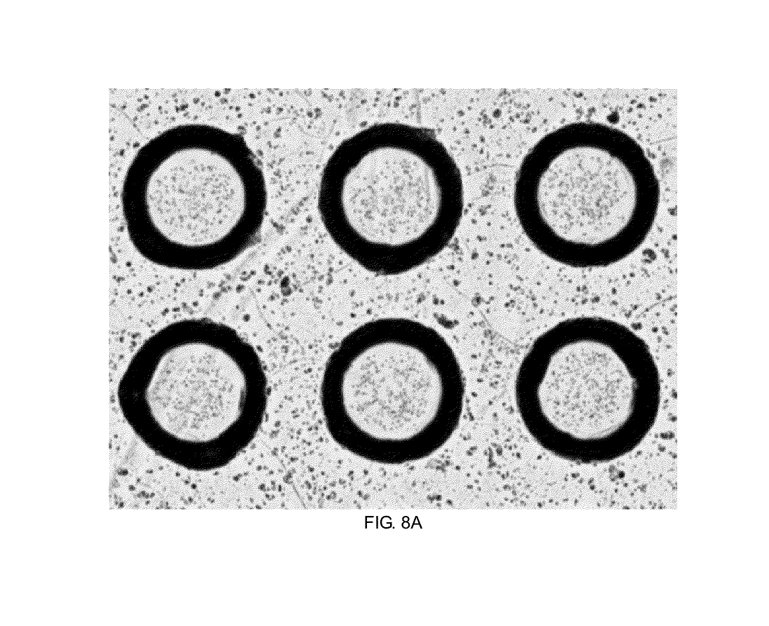

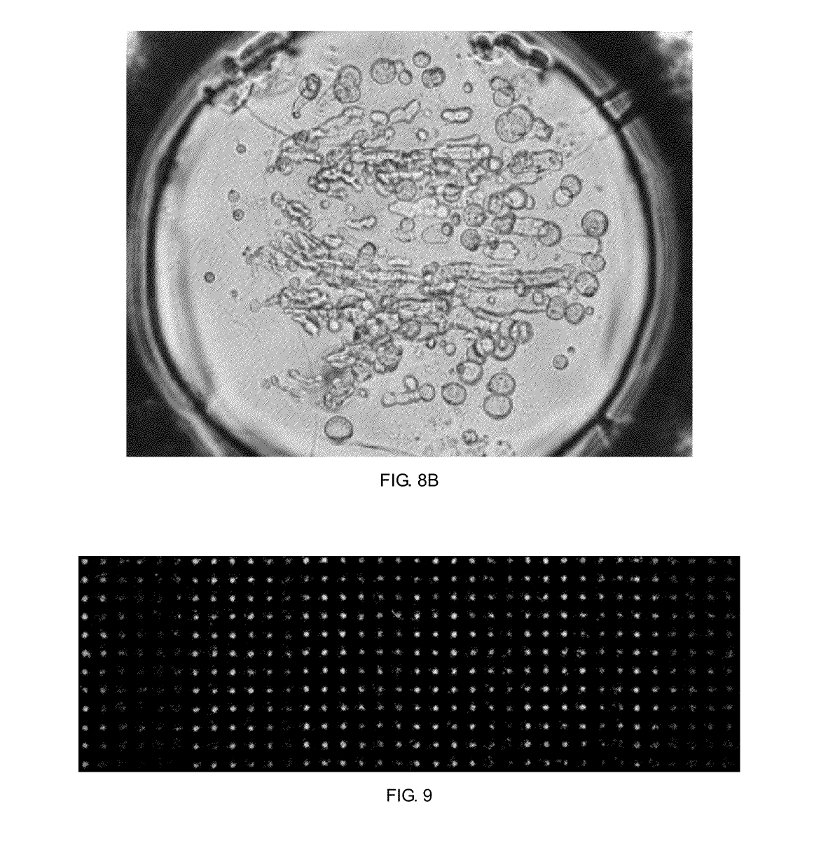

FIGS. 7A and 7B are a top image and a side image when a blue reagent puts in the well 210 of the lower substrate 200 according to the present invention and combines with the upper substrate 100. FIGS. 8A and 8B show results obtained by spotting the adhesive layer 115, the mixture of poly-L-lysine (PLL)-barium chloride to the upper substrate 100 and then, a mixture of the alginate that is a kind of biomatrices 210 and a Hep3B cell line that is a kind of hepatoma thereto and bonding them to the upper substrate 100. It can be appreciated from FIG. 8B, the Hep3B cell lines are cultured while forming the 3D structure.

FIG. 9 is a scan image showing results obtained by performing the toxicity test of a drug by using the cell chip according to the present invention.

The toxicity of a drug was tested by using the upper substrate 100 made by forming the adhesive layer of 40 nl of poly-L-lysine (PLL)-barium chloride on the spacer 130 and spotting a mixture of 40 nl of alginate-Hep3B cell line on the adhesive layer and the lower substrate 200 having the well 210. The mixture of the alginate-Hep3B cell line primarily combined the upper substrate 100 formed on the spacer with the lower substrate 200 having 800 nl of culture medium in the well 210 to gelate the alginate, thereby removing the extra barium chloride. The upper substrate 100 is separated after removing the extra barium chloride and again combines with the lower substrate 200 having 800 nl of new culture medium in the well 210, thereby culturing the Hep3B cell. When sufficient culture is completed, the upper substrate 100 is separated, combined with the lower substrate 200 having 800 nl of drug in the well 210, and cultured again, in order to test the toxicity of drug.

After the culture ends, the Hep3B cell existing on the upper substrate 100 was dyed with the fluorescent material (calcein AM and ethidium homodimer-1). As shown in FIG. 9, the living Hep3B cell was dyed with green using the calcein AM and the dead cell by the drug was dyed with red using ethidium homodimer-1.

The toxicity test on 5 drugs Chloroquine, Diclofenac, Entacapone, Mitomycin C, Tolcapone having five different concentrations was performed and the IC50 values (the concentration of drug when 50% of cells shows the growth inhibition) of the drugs obtained when the Hep3B cell was cultured in the microtiter plate according to the prior art and when the Hep3B cell was cultured in the cell chip according to the present invention, respectively, were shown in the following Table.

| | | Cell chip according to |
|---|---|---|
| Drug | Microtiter Plate(2D Hep3B cell | Invention(3D Hep3B cell |
| (compound) | monolayer in 96-wells) | culture on the chip) |
| Chliroquine | 50 uM | 10 uM |
| Diclofenac | 230 uM | 375 uM |
| Entacapone | 850 uM | 381 uM |
| Mitomycin C | 40 uM | 124 uM |
| Tolcapone | 50 uM | 171 uM |

The values shown in the above Table were obtained based on the results of culturing the drug for a day, dying it immediately after the culturing, and scanning it.

It can be appreciated that the IC50 for the drug is shown as having a value very similar to the microtiter plate.

The cell chip according to the present invention can provide the environment similar to the biomatrices environment by supplying the culture media and the reagents by being diffused into the biomaterials embedded in the biomatrices.

Further, the present invention can easily perform washing and manufacturing of the cells in the complex array form by functionally separating the lower substrate supplying the culture media or the reagent and the upper substrate formed with the biomatrices into which the biomaterials are embedded.

Further, the present invention forms the through holes on the upper substrate to provide the moving path of bubbles and air, which serve as the outlet so as not to affect the excessively supplied culture media and reagent on the adjacent biomatrices when having the array form, thereby reducing the warpage of the upper substrate.

The cell chip of the invention can be used in conjunction with an apparatus for analyzing data. For example, a cell chip scanner scans a plurality of cell chips and creates a plurality of image files. Each of the cell chips can comprise a plurality of blocks. Each of the blocks can have a plurality of spots formed by mixing a same kind of compounds having different concentrations with a same kind of enzymes. A fluorescent-intensity measuring module measures fluorescent intensities of the spots in the image files that are created by the cell chip scanner. An analyzer creates graphs in such a way as to individually conduct curve-fitting with respect to the fluorescent intensities of the spots of the blocks that are measured by the fluorescent-intensity measuring module. The analyzer creates an integrated graph in such a way as to integrate pieces of data of the blocks obtained under same test conditions and then conduct curve-fitting on the integrated data. The apparatus may further include a storage module storing test conditions. A display module may provide a test condition input window to a tester. A control module may provide the test condition input window to the tester using the display module. The control module may store input test conditions in the storage module. An input module may receive test conditions input by the tester and providing the input test conditions to the control module. The analyzer may create the graphs in such a way as to individually conduct the curve-fitting with respect to the blocks while referring to the test conditions stored in the storage module. The analyzer may include an individual curve-fitting and graph-creating module and an integrated curve-fitting and graph-creating module. The individual curve-fitting and graph-creating module may create the graphs in such a way as to individually conduct the curve-fitting with respect to the fluorescent intensities of the spots of the blocks that are measured by the fluorescent-intensity measuring module. The integrated curve-fitting and graph-creating module may create the integrated graph in such a way as to integrate the pieces of data of the fluorescent intensities of the spots of the blocks that are measured by the fluorescent-intensity measuring module under the same test conditions and then conduct curve-fitting on the integrated data. The individual curve-fitting and graph-creating module may include a data selection unit selecting a plurality of fluorescent intensities that were obtained by conducting repetitive tests on concentrations of the compounds of each of the blocks. A constant determining unit may determine a constant of a curve equation using the test results of the plurality of fluorescent intensities selected by the data selection unit. A graph creating unit may create the graph using the constant determined by the constant determining unit. The integrated curve-fitting and graph-creating module may include a data integrating unit integrating the pieces of data of the plurality of cell chips obtained under the same test conditions. A constant determining unit may determine a constant of the curve equation using the data of the cell chips integrated by the data integrating unit. A graph creating unit may create the integrated graph using the constant determined by the constant determining unit. The analyzer may include an error data control module and a report creation module. The error data control module may classify the data and sorting out an item required to be checked by the tester so as to enable the tester to delete error data. The report creation module may classify a result of the curve-fitting with respect to the integrated data and the scanned images, and create a result report, and then output the result report. The fluorescent-intensity measuring module may include a spot position detecting unit detecting positions of the spots in such a way as to calculate an average of Y-axial fluorescent-intensities and an average of X-axial fluorescent-intensities from the image files created by scanning the cell chips using the cell chip scanner. A spot boundary determining unit may determine boundaries of the spots detected by the spot position detecting unit. A spot fluorescent-intensity calculating unit may calculate a fluorescent-intensity of each of the spots after the boundaries of the spots are determined by the spot boundary determining unit.

The image files can be created by scanning a plurality of cell chips using a cell chip scanner. Fluorescent intensities of spots are measured by a fluorescent-intensity measuring module in the image files created by the cell chip scanner. Graphs are created in such a way as to individually conduct curve-fitting with respect to the fluorescent intensities of the spots of blocks that are measured by the fluorescent-intensity measuring module. An integrated graph is created in such a way as to integrate pieces of data that are related to the fluorescent intensities of the spots that are measured by the fluorescent-intensity measuring module under same test conditions and then conduct curve-fitting with respect to the integrated data.

The cell chip may be used in conjunction with a fluid discharging device and method capable of selectively discharging a relatively small amount of fluid and a relatively large amount of fluid in a wide range of viscosities from low viscosity to high viscosity. The fluid discharging device may include: a first pressure generating unit generating a first pressure for discharging a fluid; a second pressure generating unit generating a second pressure for discharging a fluid, and being controllable so as to change a magnitude of the second pressure; and a nozzle discharging the fluid pressurized by the first and second pressure generating units. The first and second pressure generating units may be connected in series with the nozzle. The first and second pressure generating units may be connected in parallel with the nozzle. The nozzle may have a storage space storing the fluid therein. The first pressure generating unit may be installed in the storage space. According to another aspect of the present invention, there is provided a fluid discharging method comprising discharging a relatively small amount of fluid and a relatively large amount of fluid by generating pressures having different magnitudes. The discharging of the relatively small amount of fluid and the relatively large amount of fluid may include discharging different amounts of fluids by simultaneously or selectively generating the pressures having the different magnitudes. The discharging of the relatively small amount of fluid and the relatively large amount of fluid may include discharging different amounts of fluids by adding or subtracting the pressures having the different magnitudes. The discharging of the relatively small amount of fluid and the relatively large amount of fluid may include quantitatively discharging fluids having different viscosities by simultaneously or selectively generating the pressures having the different magnitudes. The discharging of the relatively small amount of fluid and the relatively large amount of fluid may include quantitatively discharging fluids having different viscosities by adding or subtracting the pressures having the different magnitudes.

The cell chip may be used in conjunction with a micro droplet discharging apparatus capable of controlling the amount of droplet discharged through a single apparatus as a large amount or a small amount. The micro droplet discharging apparatus including: a pump unit generating discharging pressure; an electronic valve connected to the pump unit through a first connection pipe and controlling the discharging of a large amount of droplet; an electronic pipette connected to the electronic valve through a second connection pipe, controlling the discharging of a small amount of droplet and having droplet discharged from a distal end thereof; and a controller controlling the driving of the pump unit, the electronic valve, and the electronic pipette so as to control the amount of droplet discharged from the electronic pipette. When the controller drives the pump unit and the electronic valve, the pump unit may generate the discharging pressure and the electronic valve may allow liquid supplied from the pump unit to be discharged as the large amount of droplet from the distal end of the electronic pipette through opening and closing of the valve. When controller drives the electronic pipette, the electronic pipette may generate discharging pressure to allow liquid to be discharged as the small amount of droplet from the distal end of the electronic pipette. The pump unit may generate suction pressure to suck liquid through the electronic pipette. When the large amount of droplet is discharged to the outside, the controller may drive the pump unit and the electronic valve, such that the pump unit generates the discharging pressure and the electronic valve allows liquid supplied from the pump unit to be discharged as the large amount of droplet from the distal end of the electronic pipette through opening and closing of the valve, and stop the driving of the electronic pipette so as not to generate discharging pressure. When the small amount of droplet is discharged to the outside, the controller may drive the electronic pipette, such that the electronic pipette generates discharging pressure to allow liquid to be discharged as the small amount of droplet from the distal end of the electronic pipette, and stop the driving of the pump unit so as not to generate the discharging pressure and the driving of the electronic valve so as to be maintained in an opened state. The large amount of droplet may be about 20 nl or more; preferably about 20-10000 nl; more preferably about 25-1000 nl; preferably about 30-500 nl. The small amount of droplet may be about 5 nl or less; preferably about 2 nl or less; preferably about 1 nl or less; preferably about 0.001-1 nl; preferably about 0.1 to 1 nl. The pump unit may be a syringe pump. The syringe pump may include: an opening and closing valve having a first connection pipe connected thereto; a syringe connected to the opening and closing valve and having liquid stored therein; and a plunger moving upward in an inside of the syringe to thereby generate the discharging pressure so as to discharge the liquid to an outside of the syringe and moving downward in the inside of the syringe to thereby generate suction pressure so as to introduce the liquid to the inside of the syringe. The micro droplet discharging apparatus may further include a cleaning liquid storing tank connected to the syringe pump through a supply pipe. The electronic pipette may be a piezoelectric electronic pipette. The electronic valve may be a solenoid valve. Although the embodiments of the present invention regarding the touch panel have been disclosed for illustrative purposes, those skilled in the art will appreciate that a variety of different modifications, additions and substitutions are possible, without departing from the scope and spirit of the invention as disclosed in the accompanying claims.

While this invention has been particularly shown and described with references to preferred embodiments thereof, it will be understood by those skilled in the art that various changes in form and details may be made therein without departing from the scope of the invention encompassed by the appended claims.

1. A microarray cell chip comprising:
an upper substrate that has biomatrices encapsulating biomaterials formed on one surface thereof and one or more through-holes penetrating from one surface to the other surface thereof.

2. The microarray cell chip of claim 1, wherein at least some through-holes are positioned to provide fluid communication between the external air and a corresponding well of a lower substrate when engaged with the upper substrate such that at least a portion of the biomatrices volume reside within the well.

3. The microarray cell chip of claim 1, wherein said through-holes are positioned in near proximity to at least one spot of biomatrix encapsulating biomaterials.

4. The microarray cell chip of claim 3, wherein said spot and said through-holes are engaging one microwell from a corresponding lower substrate.

5. The microarray cell chip of claim 2, wherein said through-holes are positioned so as to allow fluid exchange of gases and/or air between surroundings and a corresponding well from a lower substrate.

6. A microarray cell chip, comprising:
an upper substrate that has biomatrices encapsulating biomaterials formed on one surface thereof and at least one through-hole penetrating from one surface to the other surface thereof; and
a lower substrate that is configured to engage with the upper substrate wherein said lower substrate contains a plurality of wells is sized to fit a plurality of biomatrices encapsulating biomaterials and at least one through-hole.

7. The microarray cell chip as set forth in claim 6, wherein the biomatrices are formed of an extracellular material or a hydrogel, preferably selected from collagen and alginate.

8. The microarray cell chip as set forth in claim 6, further comprising an adhesive layer between contacting surfaces of the biomatrices and the upper substrate.

9. The microarray cell chip as set forth in claim 6, wherein the sectional shape of the through-hole is a polygon.

10. The microarray cell chip as set forth in claim 6, wherein the through-hole is adjacently formed at an outer side of the contacting surface of the biomatrices and the upper substrate.

11. The microarray cell chip as set forth in claim 6, wherein an inlet area of the through-holes formed on one surface of the upper substrate is larger than an inlet area thereof formed on the other surface thereof.

12. The microarray cell chip as set forth in claim 6, wherein the inlet of the through-holes formed on one surface of the upper substrate is positioned on a vertical surface of the well.

13. The microarray cell chip as set forth in claim 6, wherein the biomatrices are inserted into the wells.

14. The microarray cell chip as set forth in claim 6, wherein the upper substrate and the lower substrate are provided with a protruding portion and an engaging portion engaging and/or coupling the substrates to each other.

15. The microarray cell chip as set forth in claim 6, further comprising a spacer formed on the contacting surface of the biomatrices and the upper substrate.

16. The microarray cell chip as set forth in claim 15, wherein the upper surface of the spacer is further provided with an adhesive layer encapsulating the biomatrices.

17. The microarray cell chip as set forth in claim 15, wherein the biomatrices include an amine group, and the spacer is formed of a PSMA.

18. The microarray cell chip as set forth in claim 15, wherein the through hole is adjacently formed at the outer side of the spacer.

19. The microarray cell chip as set forth in claim 15, wherein the biomatrices and the spacer are inserted into the wells.

20. The microarray cell chip as set forth in claim 15, wherein the upper surface of the spacer is concavely formed from the outer side portion to the central portion and the biomaterial is collected at the central portion.

21. The microarray cell chip as set forth in claim 15, wherein the upper surface of the spacer is formed with a plurality of concave portions and the biomaterials are collected at the concave portions.

22. The microarray cell chip as set forth in claim 15, wherein the upper substrate and the lower substrate are provided with a protruding portion and a coupling portion coupling and/or engaging the substrates to each other.

23. The microarray cell chip as set forth in claim 15, wherein the biomatrices is formed in plural, having an array form and the wells have the same array form as the biomatrices.

24. The microarray cell chip of claim 1, wherein said upper substrate contains a pillar array.

25. The microarray cell chip of claim 24, wherein said pillar array contains a plurality of columns with hollow cavity with a distal upper surface on the top of the column.

26. The microarray cell chip of claim 25, wherein said hollow cavity is sized to be capable of receiving a fiber optical imaging component.

27. The microarray cell chip of claim 1, wherein the biomatrix is a biological material.

28. The microarray cell chip of claim 27, wherein the biological material comprises Type I collagen.

29. The microarray cell chip of claim 27, wherein the biological material comprises alginate.

30. The microarray cell chip of claim 27, comprising at least 1000, at least 3000 or at least 5000 independent spots.

31. The microarray cell chip of claim 27, comprising at least 1080 independent spots.

32. The microarray cell chip of claim 27, comprising at least 560 independent spots.

33. The microarray cell chip of claim 27, wherein each of the independent spots is about 0.6 mm in size with a center-to-center distance of about 1.2 mm.

34. The microarray cell chip of claim 32, wherein the 560 independent spots are regularly spaced.
